(19) United States
(12) Patent Application Publication
(10) Pub. No.: US 2024/0033465 A1
(43) Pub. Date: Feb. 1, 2024

(54) APPARATUS FOR PROMOTING SLEEP

(71) Applicant: Blue Mountain International, LLC, Cheney, WA (US)

(72) Inventor: James Marsh, Cheney, WA (US)

(73) Assignee: Blue Mountain International, LLC, Cheney, WA (US)

(21) Appl. No.: 18/226,653

(22) Filed: Jul. 26, 2023

Related U.S. Application Data
(60) Provisional application No. 63/392,384, filed on Jul. 26, 2022.

(51) Int. Cl. A61M 21/02; H03K 3/01; A61N 2/02; A61N 2/00

(52) U.S. Cl. CPC A61M 21/02; H03K 3/01; A61N 2/02; A61N 2/006; A61M 2210/0693; A61M 2210/12

(57) ABSTRACT

A device is provided that includes a microprocessor and a transformer. The microprocessor includes a plurality of pins, the microprocessor configured to simultaneously produce a first drive signal at a first of the plurality of pins and a second drive signal at a second of the plurality of pins, the first and the drive signals having different frequencies. The transformer includes a plurality of coils, each of the plurality of coils coupled to one of the first or second pins of the microprocessor to receive one of the plurality of drive signals, the transformer therewith producing a plurality of electromagnetic fields simultaneously. The plurality of coils are configured within the device to produce a composite electromagnetic field that has a frequency that is a difference between frequencies of the plurality of electromagnetic fields

### RELATED APPLICATION

This application claims the benefit of U.S. Provisional Application No. 63/392,384, filed Jul. 26, 2023, entitled “Sleep Machine”, which is hereby incorporated by reference in its entirety.

### BACKGROUND

The present application relates to devices and corresponding methods for promoting sleep using electromagnetic fields.

Several systems have been used for promoting sleep. Pharmaceuticals, for example, will induce sleep, but are known to have side effects, may not promote deep sleep states, and reduced efficacy over time. Electronic devices have also been proposed, which attempt to soothe the user to sleep, for example, with sounds of running water and the like, but these are relatively ineffective at least when compared to pharmaceuticals. There is therefore a need for devices and corresponding methods with improved outcomes and/or with fewer or no side effects as compared to known art.

### SUMMARY OF THE INVENTION

In one aspect, a device is provided that includes a microprocessor comprising a plurality of pins, the microprocessor configured to simultaneously produce a first drive signal at a first of the plurality of pins and a second drive signal at a second of the plurality of pins, the first and the drive signals having different frequencies; and a transformer comprising a plurality of coils, each of the plurality of coils coupled to one of the first or second pins of the microprocessor to receive one of the plurality of drive signals, the transformer therewith producing a plurality of electromagnetic fields simultaneously, wherein the plurality of coils are configured within the device to produce a composite electromagnetic field that has a frequency that is a difference between frequencies of the plurality of electromagnetic fields.

In one embodiment, the microprocessor produces square-wave frequencies.

In one embodiment, the transformer is a current sensing transformer.

In one embodiment, the plurality to coils are axially aligned.

In one embodiment, the first and second frequencies are between 100 Hz and 500 Hz.

In one embodiment, the first and second frequencies are between 133 Hz and 222 Hz.

In one embodiment, a difference between the first and the second frequencies is between 1 Hz and 50 Hz.

In one embodiment, a difference between the first and the second frequencies is between 2 Hz and 10 Hz.

In one embodiment, a duty cycle of the first and second drive signals is between 30% and about 70%.

In one embodiment, the duty cycle of the first drive signal is different that the duty cycle of the second drive signal.

In another aspect, a method is provided that includes placing a device within 18 inches of a subject, the device comprising: a microprocessor comprising a plurality of pins, the microprocessor configured to simultaneously produce a first drive signal at a first of the plurality of pins and a second drive signal at a second of the plurality of pins, the first and the drive signals having different frequencies; and a transformer comprising a plurality of coils, each of the plurality of coils coupled to one of the first or second pins of the microprocessor to receive one of the plurality of drive signals, the transformer therewith producing a plurality of electromagnetic fields simultaneously, wherein the plurality of coils are configured to within the device to produce a composite electromagnetic field that has a frequency that is a difference between frequencies of the plurality of electromagnetic fields; and the method further includes producing with the device the composite electromagnetic field for a desired amount of time to affect the subject.

In one embodiment, the subject is exposed to the composite electromagnetic field for at least four hours per day.

In one embodiment, the subject is exposed to the composite electromagnetic field for at least two days.

In one embodiment, the subject is exposed to the composite electromagnetic field for a sufficient amount of time to affect the subject's sleep patterns.

In one embodiment, the subject is exposed to the composite electromagnetic field for a sufficient amount of time to affect the subject's circulation.

Additional aspects of the present invention will be apparent in view of the description which follows.

### BRIEF DESCRIPTION OF THE FIGURES

FIG. 1 is a block diagram of a device for promoting sleep according to a preferred embodiment of the devices disclosed herein.

FIG. 2A and 2B depict electromagnetic wave patterns for use in promoting sleep according to preferred embodiments herein.

### DETAILED DESCRIPTION OF THE INVENTION

Binaural beats (BB) are a phenomenon where, in one audio example, a human hears a different steady frequency (tones) in each ear. In this example, these tones are delivered by headphones to provide only 1 tone in each ear. The brain interprets a 3rd tone, that is the difference between the 2 input tones. In this example, if one ear has 100 Hz and the other ear has 125 Hz, then the brain “creates” an apparent 3rd tone of 25 Hz. Although this 3rd tone is not part of the inputs to the headphones, this interpreted tone, created by the brain.

Pulsed electromagnetic fields effects are known. Current applications include Transcranial Magnetic Stimulation (TMS). This is done in a clinical setting under medical supervision. Magnetic coils are externally located over various parts of the brain. Using various magnetic pulses, the brain functioning is altered. Some current uses include treatments for addiction, depression, anxiety, sleep among others.

The proposed application proposes a combination of PEMF, to produce a BB effect in a weak electromagnetic field to affect various tissues including circulatory and nervous system tissues with beneficial effects, and/or to promote deeper sleep, vivid dreams, reduced anxiety, reduction in pain, and other effects depending on frequencies and power chosen. In this regard, a novel device is provided that generates disparate electromagnetic waves that are combined to create a composite electromagnetic field (EMF) that stimulates sleep, reduces anxiety, moderates depression, etc. Although the device is discussed herein with respect to treating human ailments, the device is applicable to treating animal ailments, including soothing cats and dogs.

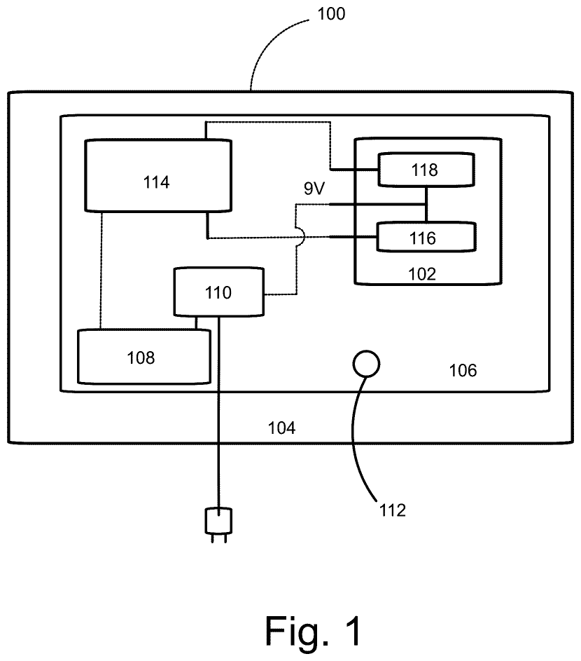

Referring to FIG. 1, device 100 includes one or more electromagnetic wave generators 102 that generate(s) a plurality of electromagnetic waves at different frequencies simultaneously, as discussed below. The wave generator(s) 102 preferably generate(s) pulsed electromagnetic fields (PEMF), which are combined to create the composite EMF.

The device 100 preferably includes one or more ABS enclosures 104, for example, having approximate dimensions 4 in.×2.75 in″×0.75″, or any dimension sufficient to enclose the components of the device therein, including a circuit board 106, such as a printed circuity board (PCB), that electrically couples the other components of the device. Enclosure 104 itself preferably contains no metal that could interfere with emission of the electromagnetic waves generated by device 100/wave generators 102.

Device 100 may include a power supply 108, which is operatively coupled to the circuit board 106, to provide power to the system components. In one embodiment, the power supply 108 includes a charging circuit 110 coupled to a rechargeable battery. The charging circuit 110 generally controls the charging of a rechargeable battery, such as a small internal non-removable LiPo battery. Device 100 may further include a power port, such as a mini-USB on one side of device 100, which provides the input power for charging the battery vis-à-vis the charging circuit 110.

Device 100 preferably includes output device 112, such as one or more LEDs that signal to the user the status of device 100 or the status of any of the functions of device 100. For example, an LED may glow green to indicate that the battery is sufficiently charged and red when the battery needs to be recharged. The device 100 may include an on/off switch to enable the functions of the device 100. In this instance, output device 112 may indicate that the device is on or off, that is, generating the composite EMF.

In one embodiment, device 100 includes a microprocessor 114, such as a microcontroller in the PIC16 family produced by Microchip Technology Inc., operatively coupled to the wave generator 102 and/or the other components of device 100 via the PCB. Microprocessor 114 preferably generates a plurality of square wave signals simultaneously that drive the wave generator(s) 102 for the generator(s) 102 to produce the composite EMF. In one embodiment, microprocessor 114 includes a plurality of pins each configured to produce independent square wave signals, which are simultaneously communicated to the wave generator(s) 102.

In one embodiment, wave generator 102 is a current sensing transformer with a plurality of axially aligned coils 116, 116. In this embodiment, each of the drive signals produced by microprocessor 114 is connected to opposite sides of the current sensing transformer. A DC signal of 4-16 VDC, preferably 9 VDC power is attached to the center tap (center pin) of the same transformer, such as the CST306-3T transformer manufactured by Triad Magnetics. The interaction of the two drive signals produced by the microcontroller causes the plurality of axially aligned coils to create competing frequencies in the transformer, which along with the 9 VDC power input creates a pulsed electromagnetic field (PEMF) by each of the coils.

In this regard, the drive signals create a composite EMF, i.e., a composite of the PEMFs, that has a smaller electrical component and a stronger magnetic field than either input signal could generate alone. The composite EMF produces characteristics of both Binaural Beats (BB) and PEMF. That is, the two different EMFs individually are perceived by the user as the difference between them, but with varying amplitudes. It is believed based on anecdotal evidence that the composite EMF affects human and animal tissues at a further distance than otherwise possible using low power and extremely low frequency (ELF) devices.

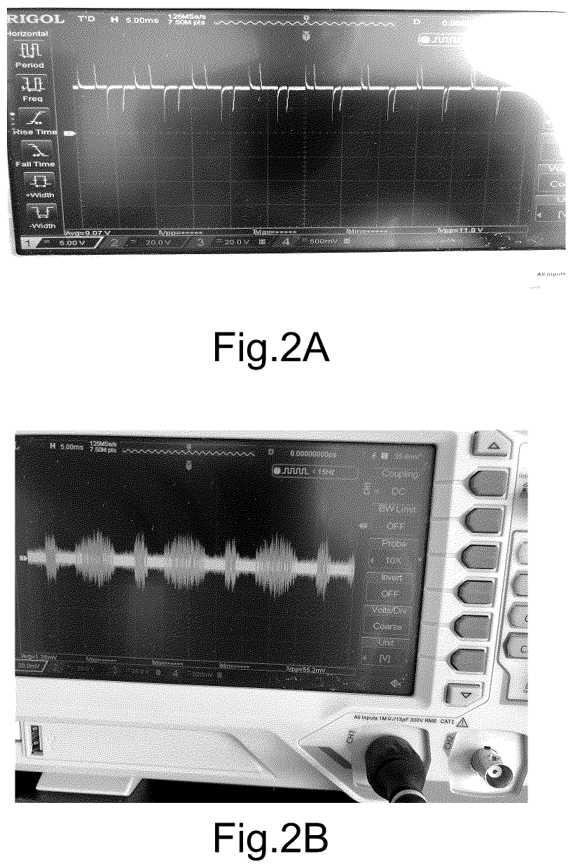

It is believed that different input frequencies can be used to produce different composite waveforms, such as those shown in FIGS. 2A-2B, that have beneficial physiological and/or psychological effects on people and animals. FIG. 2A depicts an oscilloscope showing both frequencies into the transformer and FIG. 2B depicts the composite EMC out of transformer. Both may be dynamic, constantly changing over time as the two different frequencies interact. Third, fourth, etc. frequencies with different waveforms may be added to achieve the desired EMF output. The frequencies of each of the signals are in the range of 100 Hz-500 Hz, or preferably between 136 Hz and 222 Hz with a duty cycle of 30-70%, preferably 50%. The difference between the first and the second drive signals/electromagnetic waves is between 1 Hz and 50 Hz, or preferably between 2 Hz and 10 Hz. Device 100 may include controls for users to vary the frequencies of each of the signals, to achieve the desired differential.

In one embodiment, one frequency may be 136 Hz and the other may be 141 Hz, then the BB and composite EMF/PEMF frequency is 5 Hz. This frequency differential has been shown in subjective tests to support people in getting to sleep faster, going back to sleep if they wake up at night, having more vivid dreams, and feeling more deeply rested over time.

The same or other frequency differentials may produce different results on human/animal tissue. For example, a 90 year old female with non-diabetic neuropathy that was unable to feel her feet for the past 10 years was exposed to the composite signal. Placing the device at her feet 4-5 hours per day for 4-5 days per week, she reported reduced pain, tingling on her feet, and could even feel her feet when she walked. Different pathologies responded similarly, based on what is believed to be the result of a positive effect on circulation for tissues placed near device. The proposed invention therefore uses a combination of PEMF, binaural beats in a weak electromagnetic field to affect various tissues, such as circulatory and nervous system tissues, to achieve beneficial results.

Device 100 beneficially does not make any sound or noticeable vibration. It is very low powered, drawing in the preferred embodiment less than 159 mW measured at the resister before the current sense transformer. A typical user may see improvements in 2-5 days, but it may take others over 2 weeks for the composite field to have a noticeable effect on a person's sleep pattern. The device should be kept turned on 24/7 and placed within 18 inches of the user's head to effect sleep.

In practice, device 100 is placed on or near the top of a person's bed, within about 2 feet of the user's head. Some people may attach or hang the sleep unit on the headboard of their bed, or the device may be configured as wearable. Keeping the device turned on and in proximity to the user's head 24/7 has been shown in testing to greatly improve people's sleep experience. Most people notice clear changes in their sleep experience in 2-5 days.

The composite EMF/PEMF provides a gentle, natural support for a body's nervous system. The device does not put people to sleep but rather supports people in getting to sleep quicker and sleeping deeply without the need for medications or supplements.

People report sleeping soundly and are less often disturbed by small noises. If someone does wake up during the night, they quickly fall back asleep. Reports of vivid, pleasant dreams are common. Some people report that just before waking, they have a very pleasant feeling as if they were floating on a cloud.

Initial target demographics are professions that commonly schedule people on rolling day/night shifts. These professions include, but are not limited to, health care professionals, firefighters, police, military, and industrial workers.

For example, a medical doctor working night shift reported that even when his small children were playing and making a lot of noise in the house during the day, his sleep was undisturbed. This greatly improved his ability to get needed rest before the next shift. He also stated that if he had an emergency call from work, he could more quickly wake up to deal with the situation without the usual ‘hangover’ or fog effect caused by medications or supplements. People who have been using the device for over a year report that the machine's effect does not diminish over time.

While the foregoing invention has been described in some detail for purposes of clarity and understanding, it will be appreciated by one skilled in the art, from a reading of the disclosure, that various changes in form and detail can be made without departing from the true scope of the invention.

What is claimed is:

1. A device comprising:
a microprocessor comprising a plurality of pins, the microprocessor configured to simultaneously produce a first drive signal at a first of the plurality of pins and a second drive signal at a second of the plurality of pins, the first and the drive signals having different frequencies; and
a transformer comprising a plurality of coils, each of the plurality of coils coupled to one of the first or second pins of the microprocessor to receive one of the plurality of drive signals, the transformer therewith producing a plurality of electromagnetic fields simultaneously, wherein the plurality of coils are configured within the device to produce a composite electromagnetic field that has a frequency that is a difference between frequencies of the plurality of electromagnetic fields.

2. The device of claim 1, wherein the microprocessor produces square-wave frequencies.

3. The device of claim 1, wherein the transformer is a current sensing transformer.

4. The device of claim 1, wherein the plurality to coils are axially aligned.

5. The device of claim 1, wherein the first and second frequencies are between 100 Hz and 500 Hz.

6. The device of claim 1, wherein the first and second frequencies are between 133 Hz and 222 Hz.

7. The device of claim 1, wherein a difference between the first and the second frequencies is between 1 Hz and 50 Hz.

8. The device of claim 1, wherein a difference between the first and the second frequencies is between 2 Hz and 10 Hz.

9. The device of claim 1, wherein a duty cycle of the first and second drive signals is between 30% and about 70%.

10. The device of claim 9, wherein the duty cycle of the first drive signal is different that the duty cycle of the second drive signal.

11. A method comprising:
placing a device within 18 inches of a subject, the device comprising:
a microprocessor comprising a plurality of pins, the microprocessor configured to simultaneously produce a first drive signal at a first of the plurality of pins and a second drive signal at a second of the plurality of pins, the first and the drive signals having different frequencies; and
a transformer comprising a plurality of coils, each of the plurality of coils coupled to one of the first or second pins of the microprocessor to receive one of the plurality of drive signals, the transformer therewith producing a plurality of electromagnetic fields simultaneously, wherein the plurality of coils are configured to within the device to produce a composite electromagnetic field that has a frequency that is a difference between frequencies of the plurality of electromagnetic fields; and
producing with the device the composite electromagnetic field for a desired amount of time to affect the subject.

12. The method of claim 1, wherein the subject is exposed to the composite electromagnetic field for at least four hours per day.

13. The method of claim 12, wherein the subject is exposed to the composite electromagnetic field for at least two days.

14. The method of claim 1, wherein the subject is exposed to the composite electromagnetic field for a sufficient amount of time to affect the subject's sleep patterns.

15. The method of claim 1, wherein the subject is exposed to the composite electromagnetic field for a sufficient amount of time to affect the subject's circulation.
